(19) United States
(12) Patent Application Publication
(10) Pub. No.: US 2021/0170677 A1
(43) Pub. Date: Jun. 10, 2021

(54) NOSE BRACE AND METHOD FOR MANUFACTURING A NOSE BRACE

(71) Applicant: BONE 3D, PARIS (FR)

(72) Inventors: Jérémy ADAM, PARIS (FR); Ludovic BENICHOU, PARIS (FR)

(21) Appl. No.: 17/048,517

(22) PCT Filed: Oct. 26, 2018
(86) PCT No.: PCT/FR2018/052672

§ 371 Date: Oct. 16, 2020

(30) Foreign Application Priority Data
Apr. 17, 2018 (FR) 1853336

(51) Int. Cl. B29C 64/147; B29C 64/386; A61F 5/08

(52) U.S. Cl. CPC B29C 64/147; B29L 2031/753; A61F 5/08; B29C 64/386

(57) ABSTRACT

A nose brace configured to be applied to the face of a subject and having a shaping layer in sheet form, configured to conform a nose part by bearing on a face part, the shaping layer having an inner face with a shape complementary to a digitally predefined model of the face part, the shaping layer having a shaping zone configured to conform the nose part, the shaping layer having a first upper positioning zone configured to bear on a forehead part of the face, the shaping layer having second and third lower lateral positioning zones which are each configured to bear on a face part near the nose part. The invention also relates to a method for manufacturing a nose brace.

### FIELD OF THE INVENTION

The invention relates to the technical field of rhinoplasty.

### BACKGROUND OF THE INVENTION

In particular, document US 20100042139 A1 describes a device for reshaping bones or cartilage, in particular for correcting the shape of the nose. Such a nose brace comprises a shaping member having a pressure surface intended to be placed on the skin and comprising retaining elements connected to the shaping member. The shaping member and the retaining elements cooperate so that the shaping member can come into contact with the skin while exerting a pressure force at least on the pressure surface, in order to conform the nose in an area located on or near the pressure surface. The nose brace is held on the head of a subject whose nose must be shaped, and allows the nose to be conformed without having to resort to surgery.

However, the positioning of such a nose brace is relatively loose, which may limit its efficiency. Such a brace can only conform the nose itself, and in particular cannot reposition the nose spatially relative to the rest of the face, which is possible in particular through the use of surgery. In addition, such a brace is not intended to be used after surgery, since its purpose is precisely to avoid the need for surgery.

Thus, in view of these limitations, such a nose brace offers only limited efficiency.

### SUMMARY OF THE INVENTION

This invention aims in particular to provide a nose brace, offering improved efficiency.

The invention therefore relates to a nose brace, configured to be applied to the face of a subject, characterised in that it comprises:

a shaping layer in sheet form, configured to conform a nose part by bearing on a face part, the shaping layer having an inner face with a shape complementary to a digitally predefined model of the face part,

the shaping layer having a shaping zone configured to conform the nose part,

the shaping layer having a first upper positioning zone configured to bear on a forehead part of the face,

the shaping layer having second and third lower lateral positioning zones which are each configured to bear on a face part near the nose part.

Thus, the use of a nose shaping layer, in direct contact with the subject's skin, provided with three distinct positioning zones is particularly advantageous since it can act on the nose for a certain period of time to give it a predetermined shape, i.e. the shape of a predefined model taking into account an aesthetic correction, in particular a model of the final desired nose. This shaping is carried out by bearing, in particular by mechanical restraint of the tissues in the final desired shape, imposed by the shape of the inner face of the shaping layer, which is in contact with the skin.

The three positioning zones ensure that the brace is rigorously positioned and immobilised optimally during this period, and therefore that the nose cannot move compared with the digitally predefined model. This use is particularly advantageous after nose surgery, since the shaping may prolong, improve or even correct the action of the surgery. Thus, not only can the nose brace proposed conform a nose part for a relatively long period of time without surgery, but it can also be used after surgery. This use after surgery is particularly advantageous since it offers the user the possibility of having a certain degree of control between what was achieved during and/or after the operation and the nose resulting from the digitally predefined model.

A “patient” or “subject” is generally understood to mean a living being, for example a mammal, in particular a human being.

A “sheet” is generally understood to mean an element which is relatively thin compared with its other dimensions. The sheet preferably comprises a curved inner face, in one piece, configured to bear against the nose part of the subject. Furthermore, a “shaping zone” is preferably understood to mean a zone of the sheet configured to bear against the nose part, preferably in direct contact with the skin of the nose part. The shaping zone is therefore different from a simple zone protecting the nose part such as a traditional plaster: apart from the possible protection provided, the shaping zone bears against the nose part to mechanically restrain the tissues in the final desired shape. Note that the inner face of the shaping zone has a mainly solid structure, possibly with small pores but the area of solid material is globally greater than the area of the pores, preferably much greater, for example in a ratio greater than 60/40.

The nose brace may further comprise one or more of the following characteristics, taken alone or in combination.

The shaping layer comprises stiffening ribs protruding from an outer face of the shaping layer opposite to the inner face, preferably formed integrally with the shaping layer.

The brace obtained is therefore light while retaining a rigid structure, allowing it to perform its shaping function. In other words, thanks to the stiffening ribs, a thinner shaping layer can be used. In addition, if the stiffening ribs are made integrally with the shaping layer, the nose brace is easier to manufacture.

The shaping layer comprises ventilation pores, preferably consisting of cells of dimension between 0.05 and 2 mm, more preferably between 0.5 and 1 mm, for example approximately 0.8 mm, the cells being in particular parallelepipedic or cylindrical.

Thus, the brace is lighter since it is made of less material. In addition, the patient is more comfortable, since the ventilation pores allow the skin to breathe, which avoids in particular a maceration effect due to sweating and allows drying of possible lesions or wounds. However, it is understood that, as an alternative, the shaping layer could be solid, with no pores.

The “dimension” of a cell is generally understood to mean a measurement thereof excluding thickness between the inner face and the outer face. For example, this dimension can be the diameter of a cylindrical cell, or at least the width or length of the cross-section of a parallelepipedic cell.

The thickness of the shaping layer is between 0.5 and 5 mm, preferably between 0.5 and 2 mm in the parts without stiffening ribs, for example approximately 1 mm.

Thus, the shaping layer is thick enough to conform the subject's nose, while limiting the weight of the nose brace.

The nose brace is configured to conform by bearing on at least one of the nose parts from the following list:
the nasal bone,
the septal cartilage,
a lateral nasal cartilage,
a minor alar cartilage,
an accessory nasal cartilage,
a major alar cartilage,
the fibro-adipose tissue of the wings of the nose
the septal nasal cartilage.

Thus, a shaping layer can be used to conform at least part or all of the nose, based on the digitally predefined model of the face part.

The nose brace is configured to leave exposed the parts of the face including the subject's orbicular muscle of the mouth, orifices of the nostrils and eyes.

Thus, the subject is more comfortable, the nose brace does not interfere with breathing, blowing the nose and eating.

The nose brace is made of photopolymerisable, preferably transparent material.

It is therefore easy to manufacture. In addition, when the nose brace is made of a transparent, translucent material, or a material having a colour identical or similar to that of the subject's skin, the subject is more comfortable since the visual impact of the nose brace is reduced. This is all the more advantageous since the brace is designed to perform its shaping function over a relatively long period of time, greater than 2 weeks, preferably greater than 5 weeks, for example 6 weeks.

The nose brace comprises respectively a calliper formed on the outer face of the shaping layer in each of the three positioning zones, each calliper being configured to cooperate with an attachment strap.

Thus, the means for attaching the nose brace to the subject are very easy to manufacture.

The invention also relates in particular to a nose brace assembly, comprising a nose brace of a previously defined type and straps for attachment to the subject, configured to tighten the nose brace against the subject's face, the attachment straps preferably being engaged in the callipers, for example connected together by a removable connecting member closed by a textile hook and loop system.

Thus, the means for attaching the nose brace to the subject are very easy to manufacture. The use of a removable connecting member closed by a textile hook and loop system, also known under the trademark “Velcro”, allows the subject or a user to easily remove or attach in an adjustable manner the nose brace around the subject's head.

The invention also relates in particular to a method for manufacturing a nose brace, comprising the following steps:
a step of displaying a three-dimensional image of a face part comprising a nose part to be modified, the three-dimensional image being called the initial model,
a step of modifying this three-dimensional image to define a digital model of a face part comprising a modified nose part, called the modified model,
a step of defining a digital model, called the final model, from a shape complementary to the modified model, of a nose brace comprising a shaping layer in sheet form, the shaping layer comprising a shaping zone, a first upper positioning zone and second and third lower lateral positioning zones,
a step of manufacturing, preferably by additive synthesis, a nose brace using the final model of the nose brace.

Thus, this is a simple method of obtaining a nose brace of shape adapted to the subject's face. If the nose brace is made by additive synthesis, also known as 3D printing, this manufacturing method is particularly fast and inexpensive.

The method for manufacturing a nose brace may further comprise one or more of the following characteristics, taken alone or in combination.

The nose brace is a nose brace of a previously defined type.

Thus, such a manufacturing method is particularly adapted for the manufacture of such a nose brace.

The step of defining the digital model of the nose brace comprises a step of modelling a decor.

Thus, a decor can be modelled in the digital model of the nose brace, which is then produced when manufacturing the nose brace. For example, it may be a decor comprising a colour different from that of the material of the rest of the nose brace, which may be transparent for example. Advantageously, to make the nose brace easier to wear, the decor covers the entire outer surface of the nose brace to give the appearance of a disguise. Preferably, the decor does not generate any structural modification of the nose brace. In addition, since the decor is integrated in the nose brace, in particular being made integrally with the rest of the nose brace, there is no risk of separation and in particular it does not affect the shape and/or stiffness of the nose brace.

### BRIEF DESCRIPTION OF THE DRAWINGS

It will be easier to understand the invention on reading the description below, given as an example and referring to the drawings, on which:

FIG. 1 is a front perspective view of a nose brace according to one embodiment;

FIG. 2 is a rear view of the nose brace of FIG. 1;

FIG. 3 is a side view of the nose brace of FIG. 1;

FIG. 4 is a side view of a nose, which illustrates an initial nose to be modified, a predetermined model of the final desired nose, and a superimposition of the initial nose and the final nose.

### DETAILED DESCRIPTION OF THE INVENTION

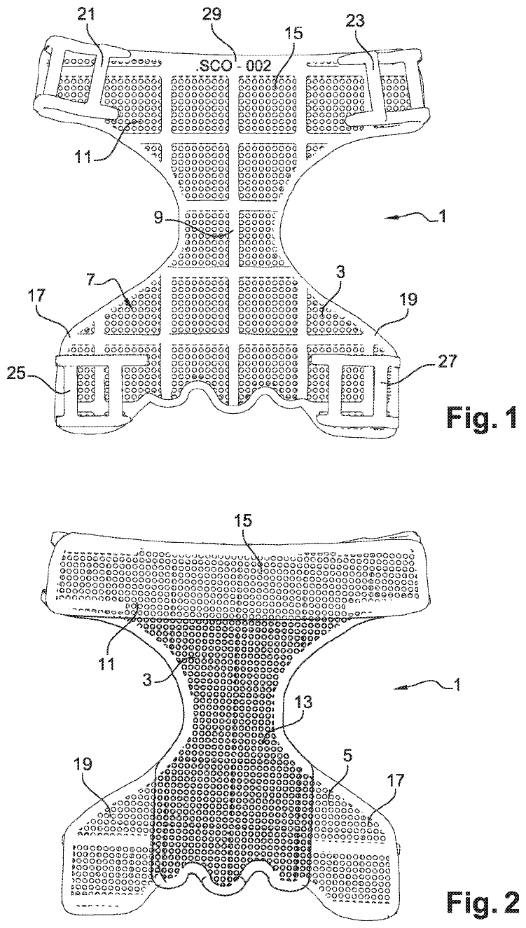

As shown on FIG. 1, a nose brace 1, configured to be applied on the face of a subject, comprises a shaping layer 3 in sheet form.

The shaping layer 3 is configured to conform a nose part by bearing on a face part.

As shown on FIGS. 1 and 2, the shaping layer 3 has an inner face 5 and an outer face 7 opposite the inner face 5. The inner face 5 has a shape complementary to a digitally predefined model of the face part and is configured to be in contact with the subject's skin. The digitally predefined model of the face part comprises a predetermined model 8 of the final desired nose as shown on FIG. 4.

The shaping layer 3 further comprises stiffening ribs 9, which protrude from the outer face 7 and ventilation pores 11, which are formed between the inner face 5 and the outer face 7.

The thickness of the shaping layer is between 0.5 and 5 mm, preferably between 0.5 and 2 mm in the parts without stiffening ribs. In this example, the thickness of the shaping layer 3 is approximately 1 mm in the parts without stiffening ribs 9, and approximately 2 mm in the parts with stiffening ribs 9.

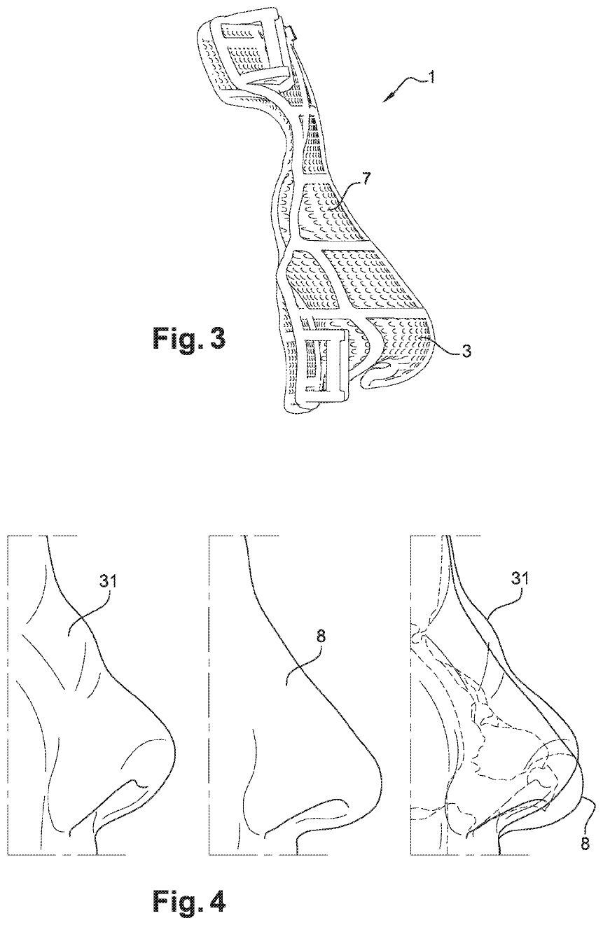

The stiffening ribs 9 are preferably made integrally with the shaping layer 3. As shown on FIGS. 1 to 3, in this example, the stiffening ribs 9 are interlaced with each other in the form of a network of meshes.

The ventilation pores 11 consist of cells of dimension between 0.05 and 2 mm, more preferably between 0.5 and 1 mm, for example approximately 0.8 mm. In this example, the ventilation pores 11 consist of cylindrical cells of approximate diameter 0.8 mm. Alternatively, the ventilation pores 11 consist of parallelepipedic cells. In addition, in this example, the ventilation pores 11 are arranged parallel to each other. Alternatively, the ventilation pores 11 are arranged substantially perpendicular to the inner face 5.

The shaping layer 3 comprises a shaping zone 13, a first upper positioning zone 15, a second lower lateral positioning zone 17 and a third lower lateral positioning zone 19.

The shaping layer 13 is configured to conform the nose part of the subject's face by bearing on at least a part of the subject's nose. Thus, the shaping zone 13 is configured to conform by bearing on at least one of the nose parts from the following list:
the nasal bone,
the septal cartilage,
a lateral nasal cartilage,
a minor alar cartilage,
an accessory nasal cartilage,
a major alar cartilage,
the fibro-adipose tissue of the wings of the nose
the septal nasal cartilage.

This shaping of all or part of the nose is in particular carried out by mechanical restraint of the tissues in the final desired shape, imposed by the shape of the inner face 5 in contact with the subject's skin, in particular by the shape of the inner face 5 in the shaping zone 13.

The first upper positioning zone 15 is configured to bear on a forehead part of the face, and extends from the upper part of the shaping zone 13. Preferably, as shown in this example, the first positioning zone 15 is wider than the shaping zone 13. This further improves the precision and ease of positioning of the nose brace 1 on the subject's face.

The second lower lateral positioning zone 17 and the third lower lateral positioning zone 19 are each configured to bear on a part of the subject's face, near the nose part, for example on a part of the subject's cheek, preferably below a zygomatic bone of the subject. Thus, the second lower lateral positioning zone 17 and the third lower lateral positioning zone 19 extend laterally on either side of the shaping zone 13, from the lower part of the shaping zone 13.

As shown on FIGS. 1 to 3, the first upper positioning zone 15, the second lower lateral positioning zone 17 and the third lower lateral positioning zone 19, are configured to position and hold in place the shaping zone 13.

In addition, as shown on FIGS. 1 to 3, the nose brace, and more particularly the shaping zone 13, the first upper positioning zone 15, the second lower lateral positioning zone 17 and the third lower lateral positioning zone 19, are configured to leave exposed the parts of the face including the subject's orbicular muscle of the mouth, orifices of the nostrils and eyes. In particular, the first upper positioning zone 15, the second lower lateral positioning zone 17 and the third lower lateral positioning zone 19, are configured so as not to obstruct the subject's eyes or lips.

In addition, since the inner face 5 of the shaping layer 3 complements a digitally predefined model of the face part, and since in particular the face part on which the first upper positioning zone 15, the second lower lateral positioning zone 17 and the third lower lateral positioning zone 19 bear is not modified, the nose brace 1 is positioned precisely on the subject's face. Since these three zones bear on parts of the face which are in particular not modified, the spatial positioning of the nose brace 1, and in particular of the shaping zone 13, is obtained precisely.

The nose brace also respectively includes a calliper 21, 23, 25, 27 formed on the outer face 7 of the shaping layer 3 in each of the three positioning zones 15, 17, 19, each calliper 21, 23, 25, 27 being configured to cooperate with an attachment strap. In particular in the example illustrated, as shown on FIG. 1, the first upper positioning zone 15 comprises two callipers 21, 23 arranged at each of its lateral ends, the second lower lateral positioning zone 17 comprises a calliper 25 and the third lower lateral positioning zone 19 comprises a calliper 27.

To form an assembly in order to press the nose brace 1 against the subject's face, attachment straps are engaged in the callipers 21, 23, 25, 27 and are in particular connected together, for example behind the subject's head, by a connecting member. This connecting member may be a removable connecting member closed with a textile hook and loop system. Thus, these attachment straps are configured to tighten in an adjustable manner and thus hold the nose brace 1 against the subject's face.

The nose brace 1 is made in particular of photopolymerisable, preferably translucent, event transparent, material. For example, the material may be a photopolymerisable resin, in particular biocompatible, more particularly sold by the company Formlabs under the name Dental LT Clear or Dental SG. More generally, the nose brace 1 is made of a material suitable for manufacturing by additive synthesis, in particular by stereolithography.

The attachment straps are for example made of textile material or elastic material. In the latter case, a connecting member is not necessarily required, since the attachment straps may for example hold the nose brace 1 against the subject's face by being stretched around the subject's head.

Lastly, the nose brace 1 comprises a decor 29. The decor 29 consists for example of letters and/or patterns, as shown on FIG. 1. On FIG. 1, the decor 29 protrudes from the outer face 7 of the shaping layer 3 of the nose brace 1, in particular from a stiffening rib 9. When the nose brace 1 is made mostly of a translucent or transparent material, the decor 29 is in particular integrated in the translucent or transparent material of the nose brace 1 with a colour different from that of the rest of the nose brace 1 made of a translucent or transparent material, so as to be visible from the outside of the outer face 7 and so that it cannot be damaged or removed without destroying the nose brace 1.

An example of a method for manufacturing the nose brace 1 will now be described.

Firstly, a three-dimensional image of a part of the subject's face is produced, the face part comprising a nose part 31 to be modified, as shown on FIG. 4. This three-dimensional image is also called the initial model. This step can for example be carried out using a three-dimensional scanner. In particular, the initial model represents not only the nose part 31 to be modified, but also at least a forehead part of the face and two cheek parts of the face extending from the nose part to be modified.

Then, a step is carried out for modifying this three-dimensional image to define a digital model of a face part comprising a modified nose part, called the modified model. All or some of this step can in particular be carried automatically using a computer program stored on a storage medium, which includes code instructions to perform this step. The illustration on the right of FIG. 4 shows a superimposition of an initial model and a modified model, to allow a user to see the modifications to be made, for example during surgery.

Then, a step is carried out for defining a digital model, from a shape complementary to the modified model, of a nose brace 1 comprising a shaping layer 3 in sheet form, the shaping layer comprising a shaping zone 13, a first upper positioning zone 15 and second and third lower lateral positioning zones 17, 19. In particular, the first upper positioning zone 15 is configured to bear on a forehead part of the face, and the second and third lower lateral positioning zones 17, 19 are each configured to bear on a face part, near the nose part, in particular on a part of the subject's cheek, preferably below a zygomatic bone of the subject. The model thus obtained is called the final model.

Optionally, this step of defining the digital model of the nose brace 1 comprises a step of modelling a decor 29.

Lastly, the nose brace 1 is manufactured from the final model, for example by additive synthesis.

If necessary, the subject undergoes surgery intended to modify the nose to give it a shape identical or at least similar to that of the final desired nose.

The nose brace 1 thus manufactured is then applied to the subject's face, and held by the attachment straps. Such a nose brace 1 is in particular much more comfortable than a plaster made on the nose after rhinoplasty surgery and, unlike a plaster, maintains or completes the shaping of the nose part. Thus, the final appearance of the subject's nose is closer, or even identical to that of the final desired nose. In addition, the time required for healing and solidification and/or shaping of the modified nose part in this final appearance is generally greater than 4 weeks, for example approximately 6 weeks or more. In contrast, the plaster requires a shorter application time, for example approximately 3 weeks, since it is only designed to protect and not to shape.

The invention is not limited to the embodiments described and other embodiments will be clearly apparent to those skilled in the art. Although the invention has been illustrated with a nose of a human subject, it can be applied to the nose of an animal subject. In addition, those skilled in the art will readily understand that the invention can also be applied to any solid body element to be modified protruding from a body surface, such as for example a mandible or cheekbone of a face of a human subject.

1. A nose brace, configured to be applied to the face of a subject, comprising:
a shaping layer in sheet form, configured to conform a nose part by bearing on a face part, the shaping layer having an inner face with a shape complementary to a digitally predefined model of the face part,
the shaping layer having a shaping zone configured to conform the nose part,
the shaping layer having a first upper positioning zone configured to bear on a forehead part of the face,
the shaping layer having second and third lower lateral positioning zones which are each configured to bear on a face part near the nose part.

2. The nose brace according to claim 1, wherein the shaping layer comprises stiffening ribs protruding from an outer face of the shaping layer opposite to the inner face.

3. The nose brace according to claim 1, wherein the shaping layer comprises ventilation pores.

4. The nose brace according to claim 1, wherein a thickness of the shaping layer is between 0.5 and 5 mm.

5. The nose brace according to claim 1, configured to conform by bearing on at least one of the nose parts from the following list:
the nasal bone,
the septal cartilage,
a lateral nasal cartilage,
a minor alar cartilage,
an accessory nasal cartilage,
a major alar cartilage,
the fibro-adipose tissue of the wings of the nose,
the septal nasal cartilage.

6. The nose brace according to claim 1, configured to leave exposed the parts of the face including the subject's orbicular muscle of the mouth, orifices of the nostrils and eyes.

7. The nose brace according to claim 1, wherein the nose brace is made of a photopolymerisable material.

8. The nose brace according to claim 1, comprising respectively a calliper formed on the outer face of the shaping layer in each of the three positioning zones, each calliper being configured to cooperate with an attachment strap.

9. A nose brace assembly, comprising a nose brace according to claim 8, and attachment straps for attachment to the subject, configured to tighten the nose brace against the subject's face.

10. A method for manufacturing a nose brace, comprising the following steps:
a step of displaying a three-dimensional image of a face part comprising a nose part to be modified, the three-dimensional image being called the initial model,
a step of modifying this three-dimensional image to define a digital model of a face part comprising a modified nose part, called the modified model,
a step of defining a digital model, called the final model, from a shape complementary to the modified model, of a nose brace comprising a shaping layer in sheet form, the shaping layer comprising a shaping zone, a first upper positioning zone and second and third lower lateral positioning zones,
a step of manufacturing, by additive synthesis, a nose brace using the final model of the nose brace.

11. The method for manufacturing a nose brace according to claim 10, wherein:
the shaping layer is configured to conform the nose part by bearing on the face part, the shaping layer having an inner face with a shape complementary to the final model,
the shaping zone is configured to conform the nose part,
the first upper positioning zone is configured to bear on a forehead part of the face, and
the second and third lower lateral positioning zones are each configured to bear on a face part near the nose part.

12. Manufacturing method according to claim 10, wherein the step of defining the digital model of the nose brace comprises a step of modelling a decor.

13. The nose brace according to claim 2, wherein the stiffening ribs are formed integrally with the shaping layer.

14. The nose brace according to claim 3, wherein the dimension is between 0.5 and 1 mm.

15. The nose brace according to claim 3, wherein the ventilation pores consist of cells of a dimension between 0.05 and 2 mm, the cells being parallelepipedic or cylindrical.

16. The nose brace according to claim 2, wherein a thickness of the shaping layer is between 0.5 and 2 mm in the parts without the stiffening ribs.

17. The nose brace according to claim 16, wherein the thickness is 1 mm.

18. The nose brace according to claim 7, wherein the photopolymerizable material is a transparent material.

19. The nose brace assembly of claim 9, wherein the attachment straps are engaged in the callipers.

20. The nose brace assembly of claim 9, wherein the attachment straps are engaged in the callipers and connected together by a removable connecting member closed by a textile hook and loop system.
